(19) United States
(12) Patent Application Publication
(10) Pub. No.: US 2025/0257117 A1
(43) Pub. Date: Aug. 14, 2025

(54) MUTANT CD24 PROTEINS AND USES THEREOF FOR PROPHYLAXIS AND TREATMENT OF CANCER

(71) Applicants: OncoC4, Inc., Rockville, MD (US); University of Maryland, Baltimore, Baltimore, MD (US)

(72) Inventors: Yang Liu, Potomac, MD (US); Pan Zheng, Potomac, MD (US); Musleh Muthana, Columbia, MD (US); Hung-Yen Chou, McLean, VA (US); Changhung Chen, Germantown, MD (US); Martin Devenport, Gaithersburg, MD (US)

(21) Appl. No.: 18/858,221

(22) PCT Filed: Apr. 20, 2023
(86) PCT No.: PCT/US2023/066022

§ 371 Date: Oct. 18, 2024

Related U.S. Application Data
(60) Provisional application No. 63/332,815, filed on Apr. 20, 2022.

(51) Int. Cl. C07K 14/705; A61K 38/00; A61K 39/00

(52) U.S. Cl. CPC C07K 14/70596; A61K 38/00; A61K 39/00; C07K 2319/02; C07K 2319/30; C07K 2319/912

(57) ABSTRACT

Provided herein are mutant CD24 proteins, RNAs, compositions thereof, and the use of the proteins and compositions in cancer therapy and as vaccines for cancer prophylaxis.

### FIELD OF THE INVENTION

The disclosure relates to CD24 protein and mRNA compositions and the use of such compositions for cancer prophylaxis and therapy.

### BACKGROUND OF THE INVENTION

CD24 is a small heavily glycosylated mucin-like glycosylphosphatidylinositol (GPI) linked cell surface protein. CD24 is expressed at higher levels on hematopoietic cells, including B cells, T cells, neutrophils, eosinophils, dendritic cells, and macrophages, as well as non-hematopoietic cells, including neural cells, ganglion cells, epithelia cells, keratinocytes, muscle cells, pancreatic cells, and epithelial stem cells. In general, CD24 tends to be expressed at higher levels in progenitor cells and metabolically active cells and to a lesser extend in terminally differentiated cells. The function of CD24 is unclear in most cell types, but diverse immunological functions of CD24 have been reported.

Although CD24 is found in many normal tissues and cell types, CD24 is overexpressed in nearly 70% of human cancers. High levels of CD24 expression detected by immunohistochemistry have been found in epithelial ovarian cancer (83%), breast cancer (85%), non-small cell lung cancer (45%), prostate cancer (48%) and pancreatic cancer (72%). CD24 is one of the most overexpressed proteins in cancer cells. CD24 expression is upregulated during tumorigenesis, suggesting its role in tumor progression and metastasis. Overexpression of CD24 in cancer has also been identified as a marker indicative of poor prognosis and a more aggressive course of the disease for cancer patients. In breast cancer, expression of CD24 is significantly higher in invasive carcinoma than benign or precancerous lesions. In non-small cell lung cancer, CD24 expression has been identified as an independent marker for the overall survival of the patient. Furthermore, in esophageal squamous cell carcinoma, CD24 overexpression is suggestive of tumor lymph node metastasis, poor tumor grade as well as reduced survival time. Similar observations were found in many other cancers including colon cancer, hepatocellular carcinoma, glioma, ovarian cancer, and prostate cancer.

Mature CD24 is a small highly glycosylated sialoglycoprotein of 31 amino acids with 16 potential O-glycosylation sites and 2 predicted N-glycosylation sites. Glycosylation is one of the most complex post-translational modifications of proteins. A shift from the normal glycosylation pathway occurs is known to occur in many cancer cells, leading to altered glycan expression and resulting in hyper-glycosylation or hypo-glycosylation of many cellular proteins. The altered glycosylation patterns found in cancer cells are the result of many contributory factors including dysregulation at the transcriptional level, dysregulation of chaperone proteins during glycosylation, and altered glycosidase and glycotransferase activities. Tumor-associated glycan changes include longer or shorter branching of N-glycans, higher or lower density of O-glycans, generation of truncated version of normal counterparts (Tn, sTn, and T antigens), and generation of unusual forms of terminal structures with sialic acid and fucose (sLea and sLex epitopes).

While CD24 has been heavily used as a prognosis marker for cancer, it has not been utilized as a neoantigen that can be a potential target for cancer therapy due to its expression on normal cell types and potential toxicity. There is a need in the art for forms of CD24 that can be used to treat and/or prevent cancer.

### SUMMARY OF THE INVENTION

Provided herein are mutant CD24 proteins that mimic one or more CD24 conformations and/or glycoforms that are uniquely presented on cancer cells but not on normal cells. The mutant CD24 protein may comprise a mature CD24 polypeptide comprising the sequence set forth in one of SEQ ID NOS: 17-19. The mature CD24 polypeptide may comprise the sequence set forth in one of SEQ ID NOS: 14-16. The mutant CD24 protein may further comprise a Fc region of a human immunoglobulin (Ig) fused at a C-terminus of the mutant CD24 protein. The Ig may be IgG1, IgG2, IgG3, IgG4, IgA, or IgM. The Ig may be IgG1, which may have the sequence set forth in SEQ ID NO: 24. The mutant CD24 protein may comprise the sequence set forth in one of SEQ ID NOS: 1-6.

The mutant CD24 protein may comprise an N-terminal CD24 signal sequence and a C-terminal CD24 glycosylphosphatidylinositol (GPI) anchor sequence, each of which may be fused to the mature CD24 polypeptide to promote proper cellular processing. The CD24 signal sequence may comprise the sequence set forth in SEQ ID NO: 42 and the GPI anchor sequence may comprise the sequence set forth in SEQ ID NO: 43. The mutant CD24 protein may comprise the sequence set forth in one of SEQ ID NOS: 7-13 and 20-23.

Further provided herein is an RNA encoding the mutant CD24 protein, which may be a messenger RNA (mRNA). The mRNA may comprise the sequence set forth in one of SEQ ID NOS: 30-35.

Also provided herein is a composition, which may be a pharmaceutical composition, comprising the mutant CD24 protein or the RNA, and a physiologically acceptable carrier or excipient.

Provided herein is a method of treating cancer or cancer prophylaxis in a patient in need thereof, which may comprise administering the mutant CD24 protein, the RNA, or the pharmaceutical composition to the patient. Further provided herein is the mutant CD24 protein, the RNA, or the pharmaceutical composition for treating cancer or cancer prophylaxis in a patient. Also provided is use of the mutant CD24 protein, the RNA, or the pharmaceutical composition in the manufacture of a medicament for treating cancer or cancer prophylaxis in a patient. The cancer may be lung cancer, ovarian cancer, breast cancer, pancreatic cancer, colon cancer, head and neck cancer, liver cancer, brain cancer, cervical cancer, ovarian cancer, renal cancer, testicular cancer, prostate cancer, or neuroblastoma. The patient may not currently have cancer but may be at high risk of developing cancer, may have completely recovered from cancer but may be at high risk of relapse, may be genetically predisposed to cancer, or may have experienced an exposure that increases the patient's cancer risk. Also provided herein is a method of optimizing a mutant CD24 protein or mRNA composition for treatment or prophylaxis of cancer.

### BRIEF DESCRIPTION OF THE DRAWINGS

FIGS. 1A-B show schematic diagrams of CD24 compositions disclosed herein. FIG. 1A shows two schematic diagrams of DNA constructs for the expression of a CD24 wild-type (SEQ ID NO: 13) and mutated (SEQ ID NO: 9) Fc fusion protein. The DNA constructs encode WT CD24 or CD24 mutant (T41>A and T51>A) fused to human heavy chain constant domains (hinge (H)—CH2-CH3). Both proteins lack the polymorphic amino acid at position 57 (based on the full-length sequence of CD24). FIG. 1B. shows two schematic drawings of WT CD24-Fc and mutated CD24-Fc fusion protein showing the dimeric structure comprising the two polypeptide components encoded by the DNA constructs shown in FIG. 1A. The T41>A and T51>A mutations of CD24 mutant Fc fusion protein unmask a cancer-specific CD24 conformation.

FIGS. 2A-C show the binding activity of anti-CD24 antibodies to different forms of CD24. FIG. 2A shows the sequence of core epitope of the mature cell surface CD24 protein having amino acid sequence of SNSGLAPN (AA42-AA49) (SEQ ID NO: 40) recognized by anti-CD24 antibodies, plus the flanking amino acids including T41 and T51 (each underlined) that are O-glycosylated (SEQ ID NO: 41), which blocks the 6373 epitope. FIG. 2B shows binding of anti-CD24 antibodies ML5, 6373 and SN3, compared to isotype control (Iso), to 293T cells transfected with wild-type (WT) and mutant CD24 proteins by FACS. FIG. 2C shows the binding of anti-CD24 antibody 6373 to 293T cells expressing wild-type (WT) and mutant CD24 proteins by FACS.

FIGS. 3A-B show capillary electrophoresis (CE)-SDS (FIG. 3A) and size exclusion-high-performance liquid chromatography (SEC-HPLC) (FIG. 3B) analysis of wild-type (WT, SEQ ID NO: 39) and mutant (mut, SEQ ID NO: 3) CD24 fusion protein expressed in CHO cells.

FIG. 4. shows ELISA data comparing binding of anti-CD24 (H3L3) and ML5 to WT CD24 fusion protein (hIgG1) and CD24^{T41>A,T51>A}fc (hIgG1). WT and mutant CD24 fusion proteins were immobilized on a plate, biotinylated H3L3 and ML5 were then added to the plate and detected with Avidin-HRP.

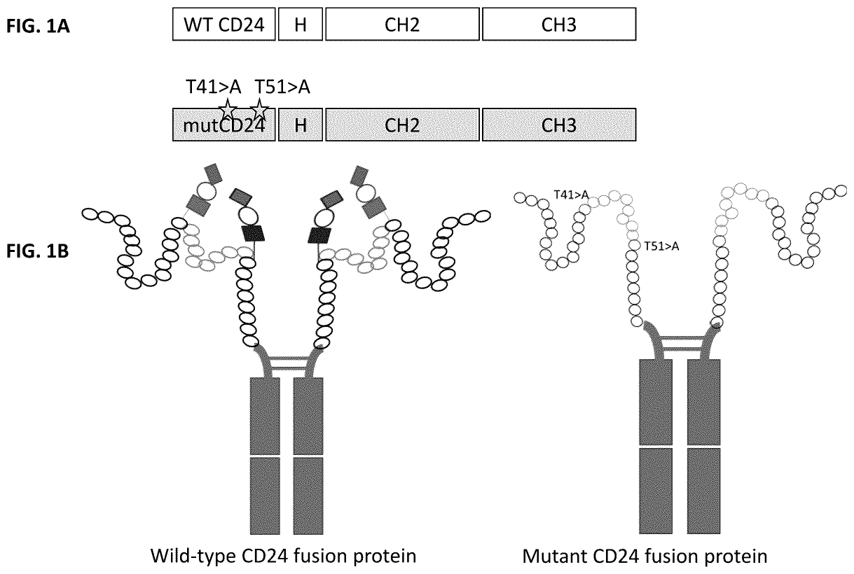

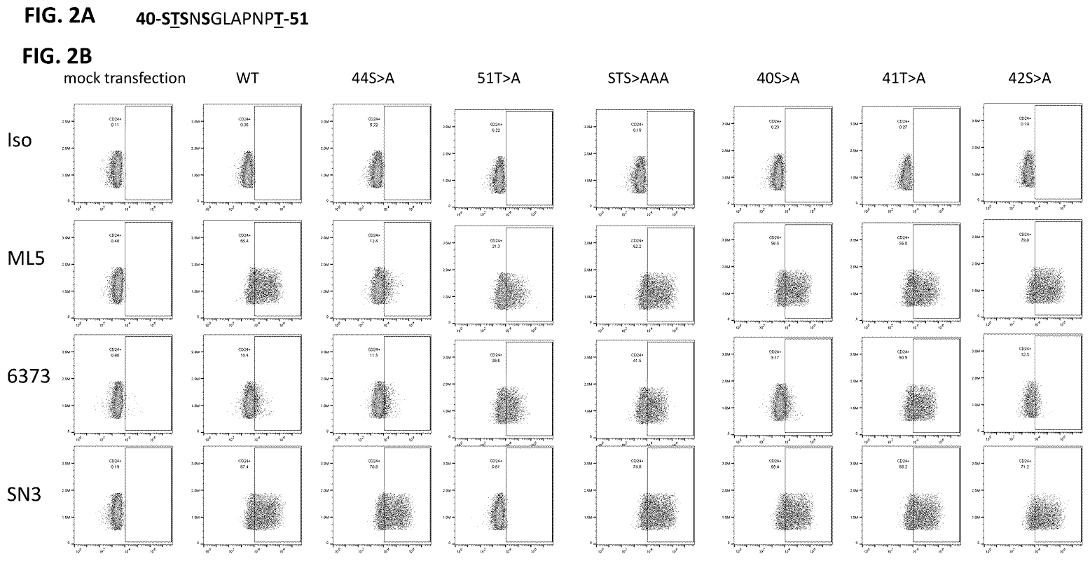

### DETAILED DESCRIPTION

Targeting of cancer expressed epitopes is a widely adopted approach for the treatment and prophylaxis of cancer. But many such epitopes do not make good drug targets because they are also expressed on normal tissues, which can lead to toxicity issues. An ideal Tumor-Specific Antigen (TSA) will have broad expression in cancer but minimal or no expression in normal or essential host organs. Attributes of less ideal but equally workable TSAs are those expressed but differentially modified in normal as compared to cancer tissues: so-called Tumor-Associated Antigens (TAA). Examples of well-characterized tumor antigens are MAGE-A3, MUC-1 and NY-ESO 1.

TSAs and TAAs can be exploited for cancer treatment or prophylaxis by targeting them directly, such as with tumor-targeting antibodies or T cells that have been genetically engineered to produce an artificial T cell receptor for use in immunotherapy, including CAR-T and TCR therapies. Alternatively, TSAs and TAAs can be used as vaccines that are administered to a person with cancer or at risk of developing cancer, to elicit an immune response to fight the cancer.

Identification of novel TSAs and TAAs is a limiting factor in the development of new or more effective cancer therapies, particularly for those cancers where tumor antigens do not currently exist. CD24 is a good cancer target for the following reasons: it is broadly over-expressed in over 70% of all human cancers and is differentially glycosylated in cancer, it appears to be oncogenic and is associated with poor prognoses in various cancers and significantly shorter patient survival, and it is a marker for cancer stem cells which can cause relapse and metastasis by giving rise to new tumors. The inventors disclose herein compositions of mutant CD24 proteins that mimic CD24 conformation and/or glycosylation patterns in cancer and their use as therapeutic and prophylactic vaccines for cancer treatment.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise. The word “about” in association with a numeric value denotes a reasonable approximation of that value. In certain cases, “about” may be construed as being within as much as 10% of the specific value with which it is associated. For example, the phrase “about 100” would encompass any value between 90 and 110.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Cancer” as used herein refers to a neoplasm or tumor resulting from abnormal uncontrolled growth of cells. As used herein, cancer explicitly includes leukemia and lymphomas. The term refers to a disease involving cells that have the potential to metastasize to distal sites. The patient may be a human.

“Mutant” as used herein means any polypeptide that contains at least one amino acid sequence alteration as compared to the amino acid sequence of the corresponding wild-type polypeptide. As used herein, an “amino acid sequence alteration” can be, for example, a substitution, a deletion, or an insertion of one or more amino acids. Mutant may also mean a protein with an amino acid sequence that is substantially identical to a referenced protein with an amino acid sequence that retains at least one biological activity. A mutant may be a derivative, analog or homolog, of a polypeptide. A mutant may also be a soluble portion of a polypeptide. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). In the context of mRNA vaccine, mutants also means change of nucleotide sequence in changing or noncoding region regardless of whether nucleotides were replaced.

“Treatment” or “treating,” may mean repressing the disease, which may involve administering a composition of the disclosure to an animal after clinical appearance of the disease.

“Prophylaxis” may mean preventing a disease, which may be administering a composition of the disclosure to an animal prior to onset of the disease.

### 2. CD24 Protein Compositions

Provided herein are mutant CD24 proteins and compositions thereof. Native, mature CD24 is a glycosylphosphatidylinositol (GPI)-anchored cell surface protein that has 31 amino acids (AA), after post-translational cleavage of the signal peptide (AA1-26) and GPI anchoring signal sequence (AA60-80) from the full-length CD24 protein. The CD24 protein may be full-length CD24 proteins and include one or more of the signal peptide and GPI anchoring signal sequence.

Two alleles of mature CD24 exist in humans, which have the WT CD24 sequence SEQ ID NO: 37 or 38, respectively. The polymorphism is at amino acid 57, and is either valine (CD24^{V}, mature sequence SETTTGTSSNSSQSTSNSGLAPNPTNATTKV (SEQ ID NO: 44)) or alanine (CD24^{A}, mature sequence SETTTGTSSNSSQSTSNSGLAPNPTNATTKA (SEQ ID NO: 45)). Anti-CD24 antibodies that bind specifically to cancer cells and tissues recognize the core epitope of the mature cell surface protein, which has the amino acid sequence SNSGLAPN (SEQ ID NO: 40) (AA42-AA49 of the full-length CD24 sequence).

Because this sequence was not altered in the cancer cells, the inventors had the insight that this epitope must have been masked in normal cells by glycan moieties anchored by amino acids surrounding the core epitope. Various mutation combinations may be introduced into the CD24 coding regions of the mature protein. The mutant CD24 protein may have a mature sequence set forth in one of SEQ ID NOS: 14-16.

(SEQ ID NO: 14)
SETTTGTSSNSSQSASNSGLAPNPTNATTK
(SEQ ID NO: 15)
SETTTGTSSNSSQSTSNSGLAPNPANATTK
(SEQ ID NO: 16)
SETTTGTSSNSSQSASNSGLAPNPANATTK

The CD24 protein may have a mutation designed to mimic a cancer-specific CD24 conformation, and may have a mature sequence set forth in one of SEQ ID NOS: 17-19, where X represents any amino acid other than S, T or N, which may anchor glycans.

(SEQ ID NO: 17)
SETTTGTSSNSSQSXSNSGLAPNPTNATTK
(SEQ ID NO: 18)
SETTTGTSSNSSQSTSNSGLAPNPXNATTK
(SEQ ID NO: 19)
SETTTGTSSNSSQSXSNSGLAPNPXNATTK

The mutant CD24 protein may also have a mutation that avoids an immune response to a polymorphic V or A at AA position 57 of the full-length CD24 protein sequence. In one example, the CD24 protein, which may be based on a WT or mutant CD24 protein sequence disclosed above, excludes the polymorphic AA at position 57. The full-length CD24 mutant sequence may have a sequence set forth in one of SEQ ID NOS: 7-12, which contain signal sequence- and GPI anchor-sequences (underlined at the N- and C-termini, respectively, of SEQ ID NO: 7) and incorporate the mutations described for the mature CD24 protein sequences SEQ ID NOS: 14-19, respectively (mutations in bold).

(SEQ ID NO: 7)
MGRAMVARLGLGLLLLALLLPTQIYSSETTTGTSSNSSQSASNSGLAPN
PTNATTKAGGALQSTASLFVVSLSLLHLYS (GPI anchored,
deleted A/V polymorphism at AA57)
(SEQ ID NO: 8)
MGRAMVARLGLGLLLLALLLPTQIYSSETTTGTSSNSSQSTSNSGLAPN
PANATTKAGGALQSTASLFVVSLSLLHLYS (GPI anchored,
deleted A/V polymorphism at AA57)
(SEQ ID NO: 9)
MGRAMVARLGLGLLLLALLLPTQIYSSETTTGTSSNSSQSASNSGLAPN
PANATTKAGGALQSTASLFVVSLSLLHLYS (GPI anchored,
deleted A/V polymorphism at AA57)
(SEQ ID NO: 10)
MGRAMVARLGLGLLLLALLLPTQIYSSETTTGTSSNSSQSXSNSGLAPN
PTNATTKAGGALQSTASLFVVSLSLLHLYS (GPI anchored,
deleted A/V polymorphism at AA57)
(SEQ ID NO: 11)
MGRAMVARLGLGLLLLALLLPTQIYSSETTTGTSSNSSQSTSNSGLAPN
PXNATTKAGGALQSTASLFVVSLSLLHLYS (GPI anchored,
deleted A/V polymorphism at AA57)
(SEQ ID NO: 12)
MGRAMVARLGLGLLLLALLLPTQIYSSETTTGTSSNSSQSXSNSGLAPN
PXNATTKAGGALQSTASLFVVSLSLLHLYS (GPI anchored,
deleted A/V polymorphism at AA57)

The full-length, WT CD24 protein sequence with the polymorphic AA at position 57 removed may have the following sequence:

(SEQ ID NO: 13)
MGRAMVARLGLGLLLLALLLPTQIYSSETTTGTSSNSSQSTSNSGLAPN
PTNATTKAGGALQSTASLFVVSLSLLHLYS (WT GPI anchored,
deleted A/V polymorphism at AA57)

In another example, the full-length, mutant CD24 protein may comprise a mutation at T41 and/or in flanking amino acids, in addition to lacking the polymorphic AA at position 57. The full-length, mutant CD24 protein may comprise a sequence set forth in one of SEQ ID NOS: 20-23 (mutations in bold).

(SEQ ID NO: 20)
MGRAMVARLGLGLLLLALLLPTQIYSSETTTGTSSNSSQATSNSGLAPN
PTNATTKAGGALQSTASLFVVSLSLLHLYS (GPI anchored,
deleted A/V polymorphism at AA57)
(SEQ ID NO: 21)
MGRAMVARLGLGLLLLALLLPTQIYSSETTTGTSSNSSQSTANSGLAPN
PTNATTKAGGALQSTASLFVVSLSLLHLYS (GPI anchored,
deleted A/V polymorphism at AA57)
(SEQ ID NO: 22)
MGRAMVARLGLGLLLLALLLPTQIYSSETTTGTSSNSSQAAANSGLAPN
PTNATTKAGGALQSTASLFVVSLSLLHLYS (GPI anchored,
deleted A/V polymorphism at AA57)
(SEQ ID NO: 23)
MGRAMVARLGLGLLLLALLLPTQIYSSETTTGTSSNSSQSTSNAGLAPN
PTNATTKAGGALQSTASLFVVSLSLLHLYS (GPI anchored,
deleted A/V polymorphism at AA57)

### 3. CD24 Fusion Protein Compositions

The CD24 proteins described herein may be fused at the N- or C-terminal end to another protein. The other protein may be a portion of a mammalian Ig protein, which may be human or mouse. The portion may comprise a Fc region of the Ig protein. The Fc region may comprise the hinge region and CH2 and CH3 domains of the Ig protein. The Ig protein may be human IgG1, IgG2, IgG3, IgG4, or IgA. The Ig protein may also be IgM, and the Fc portion may comprise the hinge region and CH3 and CH4 domains of IgM. In one example, the Fc region is human IgG1 comprising the amino acid sequence set forth in SEQ ID NO: 24.

In another example, the CD24 protein comprises a mutant mature CD24 protein sequence disclosed herein fused to the Fc region of an Ig protein, which may be the Fc region of human IgG1, particularly the one set forth in SEQ ID NO: 24. The mature CD24 fusion protein may have a sequence set forth in in one SEQ ID NOS: 1-6, which include a C-terminal IgG1 Fc region and incorporate the mature CD24 mutant proteins of SEQ ID NOS: 14-19, respectively.

(SEQ ID NO: 1)
SETTTGTSSNSSQSASNSGLAPNPTNATTKDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
(SEQ ID NO: 2)
SETTTGTSSNSSQSTSNSGLAPNPANATTKDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
(SEQ ID NO: 3)
SETTTGTSSNSSQSASNSGLAPNPANATTKDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
(SEQ ID NO: 4)
SETTTGTSSNSSQSXSNSGLAPNPTNATTKDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
(SEQ ID NO: 5)
SETTTGTSSNSSQSTSNSGLAPNPXNATTKDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
(SEQ ID NO: 6)
SETTTGTSSNSSQSXSNSGLAPNPXNATTKDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK

The CD24 proteins may also be fused at the N- or C-terminus to a protein tag, which may comprise GST, His, or FLAG, or albumin. Methods for making fusion proteins and purifying fusion proteins are well known in the art.

### 4. Mutant CD24 mRNA Compositions

Also provided herein is RNA that encodes the mutant CD24, which may be a messenger RNA (mRNA). A mature mRNA may include a 5′ untranslated region (UTR), a coding sequence (CDS), a 3′UTR, and a poly-adenosine tail (Poly-A tail). Various 5′UTRs and 3′UTRs are known in the art and can be incorporated to the mRNA sequence to enable protein expression.

In one example, the mRNA encodes a CD24 protein disclosed herein, which may be a GPI-anchored CD24 protein. The 5′UTR of mRNA may comprise a nucleotide sequence selected from the group consisting of SEQ ID NOS: 25-29, which are endogenous 5′UTRs from human CD24-coding transcript variants 1, 2, 3, 4, and 7. The coding sequence of the mutant CD24 may comprise a nucleotide sequence selected from group consisting of SEQ ID NOS: 30-35, which code for the protein in SEQ ID NOS: 7-12, respectively. In one example, the 3′UTR of the mRNA comprises the nucleotide sequence set forth in SEQ ID NO: 36, which is the endogenous 3′UTR from the human CD24 coding transcript shared between variants 1, 2, 3, 4, and 7. The poly-A tail may be any poly-A tail known in the art.

### 5. Methods of Treatment

The CD24 protein, fusion protein, and mRNA compositions described herein may be used as a vaccine to treat or prevent cancer or another abnormal proliferative disease. Provided herein is a method of such use in a patient in need thereof, which may comprise administering the CD24 protein, fusion protein, or mRNA composition, or a pharmaceutical composition comprising the foregoing, to the patient. Such molecules and pharmaceutical compositions may also be used in the manufacture of a medicament for treating or preventing cancer or another abnormal proliferative disease.

The cancer or other abnormal proliferative disease may be (but is not limited to) one or more of the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. The cancer may also be sarcoma, melanoma, or leukemia.

mRNA vaccines encoding the GPI-anchored CD24 proteins described herein would result in the encoded mutant CD24 protein being expressed on the surface of endogenous cells, which may be particularly useful for eliciting immune responses against proteins that mimic the conformation seen on cancer cells resulting in robust anti-tumor and lasting immune responses.

A cancer vaccine comprising a CD24 protein, fusion protein or mRNA molecule described herein may be used in combination with one or more other anti-tumor therapies, including but not limited to, current standard and experimental chemotherapies, hormonal therapies, biological therapies, immunotherapies, radiation therapies, or surgery. In some embodiments, the vaccine may be administered in combination with a therapeutically or prophylactically effective amount of one or more agents, therapeutic antibodies or other agents known to those skilled in the art for the treatment and/or prevention of cancer, autoimmune disease, infectious disease or intoxication. Such agents include for example, any of the above-discussed biological response modifiers, cytotoxins, antimetabolites, alkylating agents, antibiotics, or anti-mitotic agents, as well as immunotherapeutics.

The vaccine may be used in combination with one or more anti-tumor immunotherapies. The anti-tumor immunotherapy may involve one or more molecules that disrupt or enhance alternative immunomodulatory pathways (such as TIM3, TIM4, OX40, CD40, GITR, 4-1-BB, B7-H1, PD-1, CTLA-4, TIGIT, B7-H3, B7-H4, LIGHT, BTLA, ICOS, CD27 or LAG3) or modulate the activity of effecter molecules such as cytokines (e.g., IL-4, IL-7, IL-10, IL-12, IL-15, IL-17, GF-beta, IFNg, Flt3, BLys) and chemokines (e.g., CCL21) in order to enhance the immunomodulatory effects. In one example, the vaccine is combined with anti-PD-1 or anti-CTLA-4 antibodies.

### 6. Production

The CD24 protein or fusion protein vaccine may be prepared using a eukaryotic expression system. The expression system may entail expression from a vector in mammalian cells, such as Chinese Hamster Ovary (CHO) cells. The system may also be a viral vector, such as a replication-defective retroviral vector that may be used to infect eukaryotic cells. The vaccine may also be produced from a stable cell line that expresses the antibody from a vector or a portion of a vector that has been integrated into the cellular genome.

A vaccine that includes an Ig Fc fusion protein may be purified using, for example, chromatographic methods such as affinity chromatography, ion exchange chromatography, hydrophobic interaction chromatography, DEAE ion exchange, gel filtration, and hydroxyapatite chromatography. In some embodiments, fusion proteins can be engineered to contain an additional domain containing amino acid sequence that allows the polypeptides to be captured onto an affinity matrix. For example, the antibodies described herein comprising the Fc region of an immunoglobulin domain can be isolated from cell culture supernatant or a cytoplasmic extract using a protein A column. In addition, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify polypeptides.

The CD24 mRNA composition may be prepared by in vitro chemical synthesis. Large scale production of mRNA vaccines generally consists in a 1 or 2-step in vitro reaction followed by a purification platform with multiple steps that can include DNase digestion, precipitation, chromatography or tangential flow filtration. The manufacturing process can be grouped into the upstream processing, which comprises the enzymatic generation of mRNA, and the downstream processing, which includes the unit operations required to purify the mRNA product. These are complemented with LNP formulation and Fill-to-Finish steps.

### 7. Pharmaceutical Compositions

Provided herein is a pharmaceutical composition comprising the CD24 protein, fusion protein, or mRNA—which may be present at a therapeutically effective amount—and a physiologically acceptable carrier or excipient. The pharmaceutical composition may comprise a prophylactically or therapeutically effective amount of the CD24 protein, fusion protein, or mRNA, and a pharmaceutically acceptable carrier.

The term “pharmaceutically acceptable” may mean approved by a regulatory agency of the Federal or a state government, or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers may be sterile liquids, such as water and oils, including those of petroleum, animal, vegetable, or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The pharmaceutical composition, if desired, may also contain minor amounts of wetting or emulsifying agents, or pH buffering agents. These compositions may take the form of solutions, suspensions, emulsion, tablets, pills, capsules, powders, sustained-release formulations, and the like.

Generally, the ingredients of the pharmaceutical composition may be supplied either separately or mixed in unit dosage form, for example, as a dry lyophilized powder or water free concentrate in a hermetically sealed container such as an ampoule or sachet indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline may be provided so that the ingredients may be mixed prior to administration.

The pharmaceutical composition may be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The pharmaceutical composition may comprise one or more, or all of, histidine buffer, sucrose, and polysorbate 80 (PS80). In one example, the pharmaceutical composition comprises about 15, 16, 17, 18, 19, 20, 21, 22, 23, 24, or 25 mM histidine buffer. In particular, the histidine buffer concentration may be 20 mM. The pharmaceutical composition may comprise about 6, 7, 8, 9, or 10% w/v sucrose. In one example, the pharmaceutical composition comprises 8% sucrose. The pharmaceutical composition may comprise about 0.01, 0.02, or 0.03% PS80. In one example, the PS80 concentration is 0.02%. In one example, the pharmaceutical composition comprises 20 mM histidine buffer, 8% sucrose, and 0.02% w/v PS80. The pharmaceutical composition may have a pH of about 5, 5.5, or 6.0. In one example, the pH is 5.5. The pharmaceutical composition may be diluted in 0.9% sodium chloride or 5% dextrose solution before being administered to a subject.

### 8. Methods of Administration

Methods of administering the compositions and the pharmaceutical compositions thereof include, but are not limited to, parenteral administration (e.g., intradermal, intramuscular, intraperitoneal, intravenous, and subcutaneous), epidural, and mucosal (e g., intranasal and oral routes). In a specific embodiment, the composition is administered intramuscularly, intravenously, or subcutaneously. The composition may be administered by any convenient route, for example, by infusion or bolus injection, by absorption through epithelial or mucocutaneous linings (e.g., oral mucosa, rectal and intestinal mucosa, etc.) and may be administered together with one or more other biologically active agents. Administration can be systemic or local.

The CD24 proteins or fusion protein may be present in the pharmaceutical composition at about 10, 15, 20, 25, or 30 mg/mL and may be administered at a dose of about 1, 2, 3, 4 5, 6, 7, 8, 9, 10 or 20 mg/kg. A subsequent dose may be adjusted downwards from a previous dose if the subject suffers adverse events associated with administration of the antibody composition. A subsequent dose may be adjusted upwards from a previous dose if the antibody composition is not having a sufficiently strong effect against a cancer.

The CD24-encoding mRNA may be delivered using different strategies including:
i) direct injection of naked mRNA; ii) conjugation with lipid-based carriers, polymers, or peptides; and iii) via transfection of dendritic cells (DC). The direct injection of mRNA is a more cost-effective delivery alternative to DC vaccines. In vivo delivery of the naked, complexed, or encapsulated mRNA can be successfully performed by a number of administration routes such as intradermal, intramuscular, intranasal, intratumoral, intranodal or even intravenous. Using this method, a dose consisting of only a few tenths or hundreds of micrograms of mRNA (10-250 μg) is administered to each patient to trigger an immune response. The first clinical trial evaluating direct injection used naked mRNA in patients with melanoma. This approach was feasible and safe but no clinical effectiveness was observed. Self-adjuvanted RNACTIVE® vaccines is a technology developed by Cure Vac that uses a mixture of protamine-complexed and naked mRNA to improve the immunostimulatory effect of the vaccine. This technology has been successfully applied in phase I and I/II clinical trials targeting liver, prostate, lungs and melanoma cancers. New delivery approaches using lipoplexes and LNPs have been extensively used in recent clinical trials. Recent results show that both technologies can be successfully applied to treat melanoma, lymphoma, and solid tumors.

### EXAMPLES

The disclosure has multiple aspects, illustrated by the following non-limiting examples.

### Example 1

### Generation of Monoclonal Antibodies Against Hypoglycosylated CD24

On normal cells the mature extracellular CD24 protein is heavily glycosylated, whereas cancer cells have altered glycan expression resulting in hyper-glycosylation or hypo-glycosylation of many cellular proteins. It has been demonstrated that CD24 amino acids T41 and T51 both have complete O-glycosylation in normal cells, which prevents binding of the cancer-specific anti-CD24 mAb, 6373. But at least one of the T41 and T51 residues is not glycosylated in cancer cells, which allows binding by 6373. Accordingly, 6373 is a cancer-specific anti-CD24 antibody.

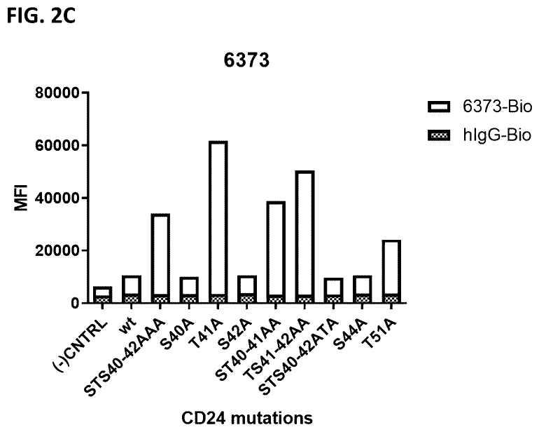

To test if the mutant CD24 mimic that hypoglycosylted CD24 on cancer cells, 293T cells were transfected with WT and mutant CD24 proteins having SEQ ID NOS: 7, 8, 13, and 20-23, and the conformation of CD24 was evaluated using antibodies that bind to all (ML5), sialylated (SN3) and hypoglycosylated (6373) forms of CD24. As shown in FIG. 2, WT CD24 expressing cells bind to ML5 and SN3, but not 6373. Mutations in T41 (T41>A, SEQ ID NO: 7), T51 (T51>A, SEQ ID NO: 8) conferred reactivity to 6373. Because the mutant residues are not required for 6373 binding, and replacing glycosylation sites unmasked the 6373 epitope, these data suggest that the 6373 epitope is shielded by glycans on both T41 and T51. Cells transfected with mutations at both residues also showed enhanced 6373 binding. Other mutations, including 40S>A (SEQ ID NO: 20), 44S>A (SEQ ID NO: 23) did not confer 6373 binding. These data suggest that CD24 on cancer cells differs from that on normal cells in glycans surrounding the 6373 core epitope, and that mutations at T41 and T51 in the CD24 core recapitulate CD24 conformation in cancer cells.

### Example 2

Fusion Proteins that Mimic Cancer-Specific CD24 Conformations

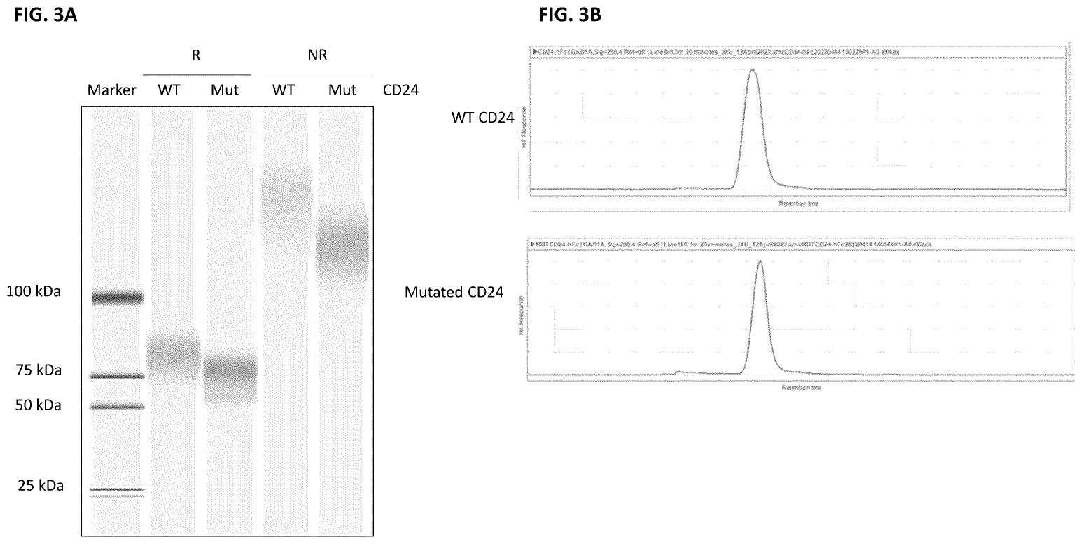

To generate a fusion protein that mimics cancer CD24-specific conformations, fusion proteins were generated, comprising the CD24 extracellular domain (mature CD24 protein) with T>A mutations at residues T41 and T51, fused to a Fc region that included the hinge region and the CH2 and CH3 domains of human IgG1 (CD24^{T41>A,T51>A}Fc) (SEQ ID NO: 3), or the wild type CD24 extracellular protein fused to the Fc region (SEQ ID NO: 39). These proteins were expressed in 293T cells and purified by affinity chromatography (FIG. 3).

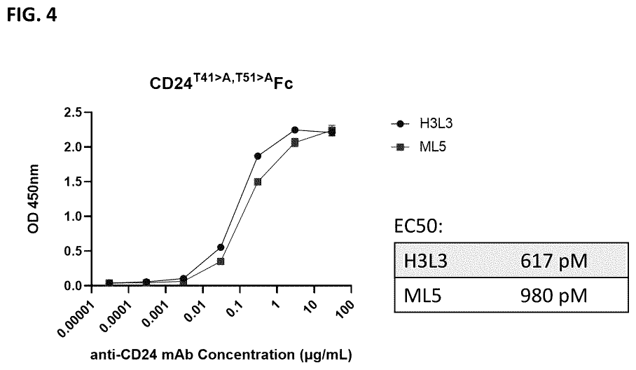

To test if the mutant fusion protein mimics the CD24 cancer conformation, binding of a humanized antibody derived from 6373, H3L3, and ML5 (which recognize all CD24 molecules regardless of its glycosylation status) to CD24^{T41>A,T51>A}Fc was measured by ELISA. As shown in FIG. 4, CD24^{T41>A,T51>A}Fc showed slightly stronger binding to H3L3 than ML5.

### Example 3

Induction of Cancer-Specific Anti-CD24 Antibodies with CD24 Vaccines

The major barrier to effective cancer vaccine is immune tolerance to CD24. To recapitulate immune tolerance, CD24 gene knock-in mice were produced. These mice will be vaccinated with a vaccine that includes mutant CD24 having one of SEQ ID NOS: 1-12, 14-23, and 30-35. After two immunizations, mouse sera will be collected and tested for binding to the vaccine and to normal vs. cancer cells using either flow cytometry or immunohistochemistry. The vaccine with the strongest antibody responses will be tested further for treatment and prophylaxis of cancer, as outlined in Example 4.

### Example 4

### Treatment or Prophylaxis of Cancer

As the first test for prophylaxis of a CD24 vaccine, human CD24 knock-in mice will be immunized two or more times and challenged with a syngeneic tumor cell line expressing human CD24. Mice that received mock immunizations will be used as control. The prophylactic activity of the vaccines will be evaluated based on growth kinetics of the human CD24-expressing tumors.

To test the effect of the vaccine on mice with genetic predisposition to cancer, transgenic mice expressing oncogenes in a tissue-specific manner will be vaccinated before the onset of tumorigenesis. One such model is TRAMP mice that express SV40 large T antigen (oncogene) under the control of the probasin promoter. Previous studies by Wang et al. showed that CD24 plays a critical role on oncogenic process in the TRAMP mice (Nat Commun 6, 5909 (2015), doi.org/10.1038/ncomms6909). The TRAMP mice express the SV40 T antigen during puberty, which initiates the process of oncogenesis in the prostate. Accordingly, vaccination will be initiated prior to puberty in order to test the prophylactic activity of the CD24 vaccines. The same cancer models can also be used to test the therapeutic effect of the vaccines, the only differences being that the vaccine will be administrated after the tumor has formed.

Once the therapeutic and prophylactic efficacy has been established, the vaccine will be evaluated for toxicity using the human CD24 knock-in mice. In one set of experiments, the mice will be vaccinated with increasing doses of vaccine, once every two weeks for 10 weeks. The mice will be monitored by potential adverse effects, including body weight loss, blood chemistry alterations, complete blood cell counts, and flow cytometric analysis of leukocytes subsets. The organs will be examined by histopathological methods. Auto-antibodies against CD24-expressing blood cells, including B cells, macrophages, neutrophils, and dendritic cells can be used as markers for autoimmunity.

Once safety is established in an animal model, the vaccine will be evaluated in human cancer patients for safety and clinical activities, including therapeutic and prophylactic activities.

1. A mutant CD24 protein comprising a mature CD24 polypeptide comprising the sequence set forth in one of SEQ ID NOS: 17-19.

2. (canceled)

3. The mutant CD24 protein of claim 1, wherein the mature CD24 polypeptide comprises the sequence set forth in one of SEQ ID NOS: 14-16.

4. The mutant CD24 protein of claim 1, further comprising a Fc region of a human immunoglobulin (Ig) fused at a C-terminus of the mutant CD24 protein.

5. The mutant CD24 protein of claim 4, wherein the Ig is IgG1, IgG2, IgG3, IgG4, IgA, or IgM.

6. The mutant CD24 protein of claim 5, wherein the Ig is IgG1.

7. The mutant CD24 protein of claim 6, wherein the Fc region has the sequence set forth in SEQ ID NO: 24.

8. The mutant CD24 protein of claim 7, wherein the mutant CD24 protein comprises the sequence set forth in one of SEQ ID NOS: 1-6.

9. The mutant CD24 protein of claim 1, further comprising an N-terminal CD24 signal sequence and a C-terminal CD24 glycosylphosphatidylinositol (GPI) anchor sequence, each fused to the mature CD24 polypeptide.

10. The mutant CD24 protein of claim 9, wherein the CD24 signal sequence comprises SEQ ID NO: 42 and the GPI anchor sequence comprises the sequence set forth in SEQ ID NO: 43.

11. The mutant CD24 protein of claim 10, wherein the mutant CD24 protein comprises the sequence set forth in one of SEQ ID NOS: 7-12.

12. An RNA encoding the mutant CD24 protein of claim 1.

13. The RNA of claim 12, wherein the RNA is a messenger RNA (mRNA).

14. (canceled)

15. The mRNA of claim 1, wherein the mRNA comprises the sequence set forth in one of SEQ ID NOS: 30-35.

16. A composition comprising the mutant CD24 protein of claim 1, and a physiologically acceptable carrier or excipient.

17. A method of treating cancer or cancer prophylaxis in a patient in need thereof, comprising administering the mutant CD24 protein of claim 1 to the patient.

18. The method of claim 17, wherein the cancer is lung cancer, ovarian cancer, breast cancer, pancreatic cancer, colon cancer, head and neck cancer, liver cancer, brain cancer, cervical cancer, ovarian cancer, renal cancer, testicular cancer, prostate cancer, or neuroblastoma.

19. The method of claim 17, wherein the method is of cancer prophylaxis.

20. The method of claim 19, wherein the patient does not currently have cancer but is at high risk of developing cancer, has completely recovered from cancer but is at high risk of relapse, is genetically predisposed to cancer, or has experienced an exposure that increases the patient's cancer risk.

21.-23. (canceled)

24. A method of treating cancer or cancer prophylaxis in a patient in need thereof, comprising administering the RNA of claim 12 to the patient.

25. The method of claim 24, wherein the cancer is lung cancer, ovarian cancer, breast cancer, pancreatic cancer, colon cancer, head and neck cancer, liver cancer, brain cancer, cervical cancer, ovarian cancer, renal cancer, testicular cancer, prostate cancer, or neuroblastoma.

26. (canceled)


SEQUENCE LISTING (45 sequences)

SEQ ID NO: 1 - protein, 257 aa, synthetic construct
SETTTGTSSNSSQSASNSGLAPNPTNATTKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 257 aa, synthetic construct
SETTTGTSSNSSQSTSNSGLAPNPANATTKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 257 aa, synthetic construct
SETTTGTSSNSSQSASNSGLAPNPANATTKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 257 aa, synthetic construct
SETTTGTSSNSSQSXSNSGLAPNPTNATTKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 257 aa, synthetic construct
SETTTGTSSNSSQSTSNSGLAPNPXNATTKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 257 aa, synthetic construct
SETTTGTSSNSSQSXSNSGLAPNPXNATTKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 79 aa, synthetic construct
MGRAMVARLGLGLLLLALLLPTQIYSSETTTGTSSNSSQSASNSGLAPNPTNATTKAGGALQSTASLFVVSLSLLHLYS

SEQ ID NO: 8 - protein, 79 aa, synthetic construct
MGRAMVARLGLGLLLLALLLPTQIYSSETTTGTSSNSSQSTSNSGLAPNPANATTKAGGALQSTASLFVVSLSLLHLYS

SEQ ID NO: 9 - protein, 79 aa, synthetic construct
MGRAMVARLGLGLLLLALLLPTQIYSSETTTGTSSNSSQSASNSGLAPNPANATTKAGGALQSTASLFVVSLSLLHLYS

SEQ ID NO: 10 - protein, 79 aa, synthetic construct
  VARIANT 41: note = X is any amino acid except for S, T or N
MGRAMVARLGLGLLLLALLLPTQIYSSETTTGTSSNSSQSXSNSGLAPNPTNATTKAGGALQSTASLFVVSLSLLHLYS

SEQ ID NO: 11 - protein, 79 aa, synthetic construct
  VARIANT 51: note = X is any amino acid except for S, T, or N
MGRAMVARLGLGLLLLALLLPTQIYSSETTTGTSSNSSQSTSNSGLAPNPXNATTKAGGALQSTASLFVVSLSLLHLYS

SEQ ID NO: 12 - protein, 79 aa, synthetic construct
  VARIANT 41: note = X is any amino acid except for S, T, or N
  VARIANT 51: note = X is any amino acid except for S, T, or N
MGRAMVARLGLGLLLLALLLPTQIYSSETTTGTSSNSSQSXSNSGLAPNPXNATTKAGGALQSTASLFVVSLSLLHLYS

SEQ ID NO: 13 - protein, 79 aa, synthetic construct
MGRAMVARLGLGLLLLALLLPTQIYSSETTTGTSSNSSQSTSNSGLAPNPTNATTKAGGALQSTASLFVVSLSLLHLYS

SEQ ID NO: 14 - protein, 30 aa, synthetic construct
SETTTGTSSNSSQSASNSGLAPNPTNATTK

SEQ ID NO: 15 - protein, 30 aa, synthetic construct
SETTTGTSSNSSQSTSNSGLAPNPANATTK

SEQ ID NO: 16 - protein, 30 aa, unidentified
SETTTGTSSNSSQSASNSGLAPNPANATTK

SEQ ID NO: 17 - protein, 30 aa, synthetic construct
  VARIANT 15: note = X is any amino acid except for S, T, or N
SETTTGTSSNSSQSXSNSGLAPNPTNATTK

SEQ ID NO: 18 - protein, 30 aa, synthetic construct
  VARIANT 25: note = X is any amino acid except for S, T, or N
SETTTGTSSNSSQSTSNSGLAPNPXNATTK

SEQ ID NO: 19 - protein, 30 aa, synthetic construct
  VARIANT 15: note = X is any amino acid except for S, T, or N
  VARIANT 25: note = X is any amino acid except for S, T, or N
SETTTGTSSNSSQSXSNSGLAPNPXNATTK

SEQ ID NO: 20 - protein, 79 aa, synthetic construct
MGRAMVARLGLGLLLLALLLPTQIYSSETTTGTSSNSSQATSNSGLAPNPTNATTKAGGALQSTASLFVVSLSLLHLYS

SEQ ID NO: 21 - protein, 79 aa, synthetic construct
MGRAMVARLGLGLLLLALLLPTQIYSSETTTGTSSNSSQSTANSGLAPNPTNATTKAGGALQSTASLFVVSLSLLHLYS

SEQ ID NO: 22 - protein, 79 aa, synthetic construct
MGRAMVARLGLGLLLLALLLPTQIYSSETTTGTSSNSSQAAANSGLAPNPTNATTKAGGALQSTASLFVVSLSLLHLYS

SEQ ID NO: 23 - protein, 79 aa, synthetic construct
MGRAMVARLGLGLLLLALLLPTQIYSSETTTGTSSNSSQSTSNAGLAPNPTNATTKAGGALQSTASLFVVSLSLLHLYS

SEQ ID NO: 24 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - RNA, 440 nt, Homo sapiens
  source 1..440: mol_type = other RNA
gcgcgcagatcgctccggacccggacaccgcctgcgaggagcgccgaccagccgggaagggttcgcgctaggcggcgcccgggtcccgtcggccagggtgagcgtccggcccgcgtccgcgccacgcccgccgcgttcccctttcctctgcggcgggccgagagataaccctgcccgaggggtcccggcgcccgccccccacgcggtcgcactggaattcgcagcccctctcgggtccccggggcgcattttgcagtctgagtggcaatgcacttgctccaggacaggcggctaccccgccgcagcggaggcgcggacttttcttttggggggtctcgccggctcgccgcgctccccaccttgcctgcgcccgcccggagccagcggttctccaagcacccagcatcctgctagacgcgccgcgcaccgacggaggggac

SEQ ID NO: 26 - RNA, 367 nt, Homo sapiens
  source 1..367: mol_type = other RNA
agtttgcagcgtcaggcagcgggtctgcgcccagcgagcggctccccagctcctgggaagatgcggacccgggacgcccccgtgagctcactgcgcctggctgacacgaggcgctcacagaacaaagcaagggcttcggggagggcgcggccgcggggccgagcgcgcagatcgctccggacccggacaccgcctgcgaggagcgccgaccagccgggaagggttcgcgctaggcggcgcccgggtcccgtcggccagggtctcgccggctcgccgcgctccccaccttgcctgcgcccgcccggagccagcggttctccaagcacccagcatcctgctagacgcgccgcgcaccgacggaggggac

SEQ ID NO: 27 - RNA, 222 nt, Homo sapiens
  source 1..222: mol_type = mRNA
tgctcttgttgcccaggctggaatgcaatggcctgatctctgctcactgcaacctccacctctcaggttcaagctattctcctgcctcagcctcccaagtagctgggattacaggtctcgccggctcgccgcgctccccaccttgcctgcgcccgcccggagccagcggttctccaagcacccagcatcctgctagacgcgccgcgcaccgacggaggggac

SEQ ID NO: 28 - RNA, 154 nt, Homo sapiens
  source 1..154: mol_type = mRNA
ctttttaaacaaaccccggttccgggctgaagccgaggcggggaccctctcctgacgctgcgctctctgcgaggcaacctttcacgtccatcgcttgtcatcttaggatccccatgttattttagcccagattggctagttctggggaggcagc

SEQ ID NO: 29 - RNA, 83 nt, Homo sapiens
  source 1..83: mol_type = mRNA
accttgcctgcgcccgcccggagccagcggttctccaagcacccagcatcctgctagacgcgccgcgcaccgacggaggggac

SEQ ID NO: 30 - RNA, 240 nt, synthetic construct
  source 1..240: mol_type = other RNA
atgggcagagcaatggtggccaggctcgggctggggctgctgctgctggcactgctcctacccacgcagatttattccagtgaaacaacaactggaacttcaagtaactcctcccagagtgcctccaactctgggttggccccaaatccaactaatgccaccaccaaggctggtggtgccctgcagtcaacagccagtctcttcgtggtctcactctctcttctgcatctctactcttaa

SEQ ID NO: 31 - RNA, 240 nt, synthetic construct
  source 1..240: mol_type = other RNA
atgggcagagcaatggtggccaggctcgggctggggctgctgctgctggcactgctcctacccacgcagatttattccagtgaaacaacaactggaacttcaagtaactcctcccagagtacttccaactctgggttggccccaaatccagccaatgccaccaccaaggctggtggtgccctgcagtcaacagccagtctcttcgtggtctcactctctcttctgcatctctactcttaa

SEQ ID NO: 32 - RNA, 240 nt, synthetic construct
  source 1..240: mol_type = other RNA
atgggcagagcaatggtggccaggctcgggctggggctgctgctgctggcactgctcctacccacgcagatttattccagtgaaacaacaactggaacttcaagtaactcctcccagagtgcctccaactctgggttggccccaaatccagccaatgccaccaccaaggctggtggtgccctgcagtcaacagccagtctcttcgtggtctcactctctcttctgcatctctactcttaa

SEQ ID NO: 33 - RNA, 240 nt, synthetic construct
  source 1..240: mol_type = other RNA
  variation 121..123: product = encodes any amino acid except for S, T, or N
atgggcagagcaatggtggccaggctcgggctggggctgctgctgctggcactgctcctacccacgcagatttattccagtgaaacaacaactggaacttcaagtaactcctcccagagtnnntccaactctgggttggccccaaatccaactaatgccaccaccaaggctggtggtgccctgcagtcaacagccagtctcttcgtggtctcactctctcttctgcatctctactcttaa

SEQ ID NO: 34 - RNA, 240 nt, synthetic construct
  source 1..240: mol_type = other RNA
  variation 151..153: product = encodes any amino acid except S, T, or N
atgggcagagcaatggtggccaggctcgggctggggctgctgctgctggcactgctcctacccacgcagatttattccagtgaaacaacaactggaacttcaagtaactcctcccagagtacttccaactctgggttggccccaaatccannnaatgccaccaccaaggctggtggtgccctgcagtcaacagccagtctcttcgtggtctcactctctcttctgcatctctactcttaa

SEQ ID NO: 35 - RNA, 240 nt, synthetic construct
  source 1..240: mol_type = other RNA
  variation 121..123: product = encodes any amino acid except for S, T, or N
  variation 151..153: product = encodes any amino acid except for S, T, or N
atgggcagagcaatggtggccaggctcgggctggggctgctgctgctggcactgctcctacccacgcagatttattccagtgaaacaacaactggaacttcaagtaactcctcccagagtnnntccaactctgggttggccccaaatccannnaatgccaccaccaaggctggtggtgccctgcagtcaacagccagtctcttcgtggtctcactctctcttctgcatctctactcttaa

SEQ ID NO: 36 - RNA, 363 nt, Homo sapiens
  source 1..363: mol_type = mRNA
gagactcaggccaagaaacgtcttctaaatttccccatcttctaaacccaatccaaatggcgtctggaagtccaatgtggcaaggaaaaacaggtcttcatcgaatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaataagaatagaaataaaggatgggattgtggaatgga

SEQ ID NO: 37 - protein, 80 aa, Homo sapiens
MGRAMVARLGLGLLLLALLLPTQIYSSETTTGTSSNSSQSTSNSGLAPNPTNATTKAAGGALQSTASLFVVSLSLLHLYS

SEQ ID NO: 38 - protein, 80 aa, Homo sapiens [sequence omitted]

SEQ ID NO: 39 - protein, 257 aa, synthetic construct
SETTTGTSSNSSQSTSNSGLAPNPTNATTKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 8 aa, Homo sapiens
SNSGLAPN

SEQ ID NO: 41 - protein, 12 aa, Homo sapiens
STSNSGLAPNPT

SEQ ID NO: 42 - protein, 26 aa, Homo sapiens
MGRAMVARLGLGLLLLALLLPTQIYS

SEQ ID NO: 43 - protein, 23 aa, Homo sapiens
AGGALQSTASLFVVSLSLLHLYS

SEQ ID NO: 44 - protein, 31 aa, Homo sapiens
SETTTGTSSNSSQSTSNSGLAPNPTNATTKV

SEQ ID NO: 45 - protein, 31 aa, Homo sapiens
SETTTGTSSNSSQSTSNSGLAPNPTNATTKA